(19) United States
(12) Patent Application Publication
(10) Pub. No.: US 2021/0052838 A1
(43) Pub. Date: Feb. 25, 2021

(54) SYSTEM AND VENTILATOR FOR NONINVASIVE DETECTION OF INFECTION DURING VENTILATION

(71) Applicant: Loewenstein Medical Technology S.A., Luxembourg (LU)

(72) Inventor: Matthias SCHWAIBOLD, Karlsruhe (DE)

(21) Appl. No.: 16/995,956

(22) Filed: Aug. 18, 2020

(30) Foreign Application Priority Data
Aug. 20, 2019 (DE) 102019005846.1

(51) Int. Cl. A61M 16/00

(52) U.S. Cl. CPC A61M 16/0051; A61M 16/0003; A61M 16/024; A61M 2205/3306; A61M 2205/3553; A61M 2016/0027; A61M 2016/003; A61M 2205/3368

(57) ABSTRACT

A system for noninvasive measurement of body temperature during ventilation, comprising a temperature sensor, a ventilator, an interface between ventilator and temperature sensor, a processing unit, a data storage unit, a user interface, an interface to a remote alarm, a remote alarm, a ventilation tube having a patient interface, and a respiratory gas sensor. The ventilator is configured to identify phases of inspiration and expiration by means of the respiratory gas sensor and to convey respiratory gas during inspiration and expiration. The system is configured to ascertain a sensor signal by means of the temperature sensor and the processing unit and to check whether the sensor signal is representative of body temperature and to process and/or to save such body temperature signals and to compare them with saved rules or threshold values.

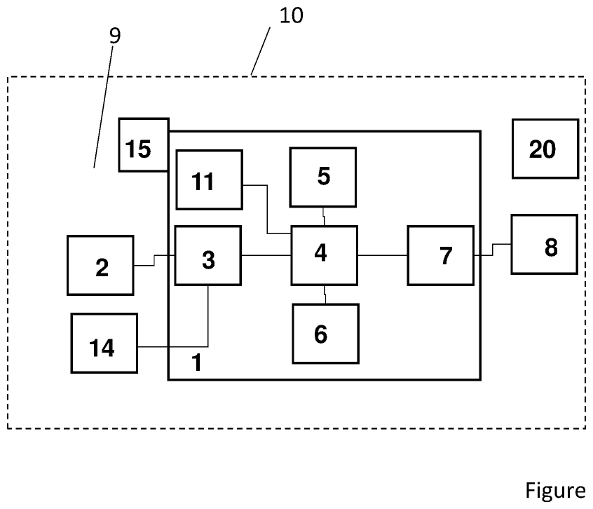

### CROSS-REFERENCE TO RELATED APPLICATIONS

The present application claims priority under 35 U.S.C. § 119 of German Patent Application No. 102019005846.1, filed Aug. 20, 2019, the entire disclosure of which is expressly incorporated by reference herein.

### BACKGROUND OF THE INVENTION

### 1. Field of the Invention

The invention relates to a system and a ventilator for noninvasive detection of infection during ventilation, comprising at least one sensor designed for detection of infection.

### 2. Discussion of Background Information

For patients who are ventilated in a domestic setting or in a hospital, infections can be detected only with difficulty or, in many cases, only at a late stage. Especially in the case of chronic stable pulmonary diseases (COPD, asthma, NMD), respiratory tract infections are frequently triggers for a decompensation/exacerbation, which makes an additional treatment necessary.

Infections can be identified by a rise in body temperature.

There are various devices and methods for measuring the body temperature of humans. In the case of invasive measurement methods, temperature sensors are introduced into the body via body openings and the core temperature is gathered from the signal of the temperature sensors. However, such measurement methods are susceptible to artifacts and are not tolerated well in the long term.

It is precisely during ventilation that it is difficult to perform temperature measurement using conventional methods.

An early detection of a respiratory tract infection during ventilation before a severe exacerbation occurs is clearly desirable.

In view of the foregoing, it would be advantageous to have available a system for noninvasive detection of infection, comprising at least one sensor which is of a simplest and most cost-effective possible construction and which is integrated into the ventilation system and is designed for detection of infection.

### SUMMARY OF THE INVENTION

The present invention provides a system for the noninvasive detection of infection during ventilation, comprising at least one sensor designed for detection of infection, a ventilator, an interface between ventilator and sensor, a processing unit, a data storage unit, a user interface, an interface to a remote alarm, a remote alarm, a ventilation tube having a patient interface, and at least one respiratory gas sensor for determination of pressure and/or flow of respiratory gas.

The ventilator is preferably configured and designed to identify phases of inspiration and expiration by means of the respiratory gas sensor and to convey respiratory gas during inspiration and expiration. The system is preferably configured to ascertain a sensor signal by means of the sensor and the processing unit, at least in phases during inspiration and expiration, and to check whether the sensor signal is representative of body temperature or an infection and to process and/or save such sensor signals and to compare them with stored rules or threshold values.

The present invention further provides a ventilator in a system according to the invention or for use with a system according to the invention.

What is preferred according to the present invention is that the at least one sensor has connected thereto a transmitter unit for transmitting the sensor values measured.

According to the invention, the sensor is designed to detect an infection or inflammation. The sensor is designed to detect parameters or measurement values which are indicative of an infection or inflammation. To this end, the sensor can be designed as a temperature sensor or an activity sensor as an alternative to detecting pathogens in the air, a rise in the average heart rate, a change in skin impedance, rapid weight loss, or further parameters which are indicative of an infection or inflammation.

According to an advantageous embodiment, the system may also be characterized in that at least one temperature sensor is formed by a thermistor.

According to an alternative advantageous embodiment, the system may also be characterized in that at least one temperature sensor is formed by an optical sensor.

According to one aspect of the invention, it is proposed that at least one temperature sensor is provided in the tube or the patient interface for measurement of the temperature of inspiration flow and/or expiration flow.

According to a further aspect of the invention, it is proposed that at least one temperature sensor is arranged adjacent to the body or skin of the patient.

According to an additional aspect of the invention, it is proposed that at least one temperature sensor is provided in the region of the patient interface and is arranged such that the sensor is arranged adjacent to the body or skin of the patient, for example fixed by an adhesive surface or a magnet or a clip to the forehead support of the patient interface or to the banding of the patient interface or to the bead of the patient interface or to the body of the patient interface or to a finger, the forehead or the ear lobe of the patient.

According to an alternative aspect of the invention, it is proposed that at least one temperature sensor is arranged away from the body or the skin of the patient, for example in the form of a thermal imaging camera.

According to a further advantageous embodiment, an activity sensor is also provided in addition to the temperature sensor.

According to an alternative or additional embodiment, the sensor is designed as an activity sensor which can be designed to detect an infection from the decrease in physical activity in comparison with an earlier comparative period.

According to an additional advantageous embodiment, the interface between the ventilator and the temperature sensor conducts either data only (temperature sensor then has its own power source) or data and temperature-sensor power supply. For example, the system is also characterized in that the interface is realized as a cable, for example USB, IC, serial, light guide, LAN, ESATA, . . . .

In addition or as an alternative, the system may also be characterized in that the interface is realized wirelessly, for example as Bluetooth, WIFI, low-power standard.

In addition or as an alternative, the system may also be characterized in that the transmission of sensor data via the interface takes place at least daily, preferably at least hourly, particularly preferably at least 1× to 60× per minute.

Advantageously, the system may also be characterized in that the processing unit is designed and configured to detect whether a sensor signal is present and whether a sensor signal represents a temperature signal.

Advantageously, the system may also be characterized in that the processing unit compares the sensor signal with a stored value range which represents typical body temperature signals, for example in the range between 33 and 45 degrees Celsius, preferably between 35 and 44 degrees Celsius.

According to a further advantageous embodiment, the processing unit checks the sensor signal for recurring maximum and minimum temperature signals and classifies at least the maximum temperature signals as body temperature signals.

According to an advantageous embodiment, the processing unit compares the sensor signal of the temperature sensor with the sensor signal of the activity sensor.

The system may, for example, alternatively or additionally also be characterized in that the processing unit compares the sensor signal of the temperature sensor with the sensor signal of the activity sensor and processes as nocturnal body temperature signals those sensor signals of the temperature sensor that are registered simultaneously with a low sensor signal of the activity sensor over a period of at least 3 hours.

The system may, for example, alternatively or additionally also be characterized in that the processing unit is designed and configured to process the temperature signal, for example by conversion of the units, smoothing, . . . , and to save it in the data storage unit. The conversion of units can, for example, be a conversion of sensor signals which are present analogically in the form of a voltage or digitally as a bit value and are converted into a unit associated with the physiological variable measured. In the case of a measured temperature, this can, for example, be degrees Celsius or degrees Fahrenheit or a similar variable or unit associated with the temperature. A smoothing of the units can, for example, be achieved by an averaging or a weighted averaging of the signals over a period. The removal of outlier signals can be used as a smoothing measure, too. What can be regarded as outlier signals are, for example, individually occurring signals or values which are not within a plausible range of a body temperature, or the value deviates too far from the previous measurement values. If outlier signals occur more frequently and/or directly one after another over time, they can also be classified as nonoutlier signals and possibly trigger a corresponding alarm.

The system may alternatively or additionally also be characterized in that the processing unit is designed and configured to compare the temperature signal with stored alarm thresholds, said alarm thresholds representing an excessively high temperature or low temperature or the loss of the data connection or a deterioration of the sensor or an expiration of the service life of the sensor or excessively low battery level of the sensor.

The system may, for example, alternatively or additionally also be characterized in that the processing unit is designed and configured to compare the temperature signal with stored alarm thresholds and, in the event of an alarm threshold for increased body temperature being exceeded, the processing unit may be designed and configured to control at least one ventilation parameter of the ventilator such that the result is a ventilation matched to the increased body temperature, thus ensuring an optimized relief or support for the patient.

Advantageously, the system may also be characterized in that the processing unit is designed and configured to compare the temperature signal with stored alarm thresholds and, in the event of an alarm threshold for an increased or decreased body temperature being exceeded (temperature alarm), the processing unit is designed and configured to change at least one threshold for a ventilation parameter alarm (ventilation alarm). For example, the ventilation alarm may become more sensitive at increased body temperature.

According to the invention, the system may also be characterized in that the processing unit is designed and configured to compare the temperature signal with stored alarm thresholds and, in the event of an alarm threshold for an increased or decreased body temperature being exceeded (temperature alarm), the processing unit may be designed and configured to request, in an automated manner, feedback from the patient, said feedback being a digital response from the patient that represents a current state.

Advantageously, the system may also be characterized in that the patient's response is input by said patient via the end-user interface.

According to one aspect of the invention, the processing unit may be designed and configured to compare the temperature signal with stored alarm thresholds and, in the event of an alarm threshold for an increased or decreased body temperature being exceeded (temperature alarm), the processing unit may be designed and configured to output recommended actions to the patient via the end-user interface or a loudspeaker.

Advantageously, the system may also be characterized in that the processing unit is designed and configured to form statistics, for example containing max/min/mean values/proportion with value >x for defined time intervals, for example per hour or per day, and to save them in the data storage unit. Triggering of an alarm can, too, be caused on the basis of said statistics. If the temperature value is above a value for a certain time, this can serve as the trigger for the alarm. Gradations could also be introduced here. For example, the temperature value can be above 38 degrees Celsius for a period, for example for one hour, without an alarm being triggered; this can be far less, for example just one minute or less, for higher temperature values, for example above 40 degrees Celsius.

Lastly, according to a further aspect of the teaching, the processing unit may be designed and configured to process signals of the temperature sensor and signals of the respiratory gas sensor and signals of the activity sensor and to plausibilize signals of the temperature sensor by a comparison with signals of the respiratory gas sensor and/or signals of the activity sensor.

In particular, the system may also be characterized in that the data storage unit is designed and configured to record, in a retrievable manner, the course of the temperature signal and preferably also the statistics which were calculated and alarms which occurred.

The system may alternatively also be characterized in that the user interface, for example as display, LEDs, loudspeaker, projection screen, is designed and configured to depict the temperature signal as temperature and, optionally, to also depict statistics, alarms, courses. The system may alternatively or additionally also be characterized in that the user interface is designed and configured to generate feedback as to whether a temperature sensor has been connected correctly and is providing values or not, preferably in the form of a graphical and acoustic output, for example warning tone in the event of an alarm.

The system may alternatively or additionally also be characterized in that the interface to the remote alarm is designed and configured to conduct either data only (remote alarm has its own power source) or data and power in relation to the remote alarm.

The system may alternatively or additionally also be characterized in that the interface to the remote alarm is designed as a cable (USB, IC, serial, light guide, LAN, ESATA, I2C, analog lines with a variable voltage level, . . . ) or wirelessly (Bluetooth, WIFI, low-power standard, GSM, UMTS, LTE, NB-IOT, Lora, Sigfox, G mobile communications).

The system may alternatively or additionally also be characterized in that the interface to the remote alarm preferably consists of a network, for example hospital network, Internet, VPN network, at least in part or is integrated in a network.

The system may alternatively or additionally also be characterized in that the interface to the remote alarm is configured and designed for at least daily, preferably at least hourly, preferably at least 1× to 60× per minute, transmission; preferably also triggered by the occurrence of an alarm.

Advantageously, the system may also be characterized in that the remote alarm is designed and configured to be, for example, executed on a computer screen, for example in a browser in the case of Web-based telemonitoring or controlled by local software which can communicate with the ventilator, or on a handheld device such as a smartphone, notebook or tablet, likewise either in a browser or controlled by local SW (software), or by an acoustic signal generator (nurse call) or in the form of a message, for example SMS, messenger, e-mail.

Further advantageously, the system may also be characterized in that the system comprises a humidifier which is configured and designed to warm and humidify respiratory gas, account being taken of body temperature signals of the temperature sensor at least from time to time or in part for the control of the humidifier in order to warm and humidify the respiratory gas according to the body temperature signal.

Additionally advantageously, the system may also be characterized in that the system moreover comprises a temperature sensor which measures the ambient temperature and the processing unit is designed and configured to compare the temperature signal of the sensor with the temperature signal of the sensor.

Further advantageously for example, the system may also be characterized in that the system moreover comprises a temperature sensor which measures the ambient temperature and the processing unit is designed and configured to emit an alarm, for example a remote alarm, in the event of an alarm threshold for an increased or decreased ambient temperature being exceeded.

Preferably, the system may also be characterized in that the system moreover comprises a temperature sensor which measures the ambient temperature and an air-conditioning unit for the ambient temperature and the processing unit is designed and configured to control the air-conditioning unit, in the event of an alarm threshold for an increased or decreased ambient temperature being exceeded, such that the ambient temperature is adjusted. The ventilator may be a clinical ventilator or a ventilator for domestic ventilation or CPAP or bilevel therapy or a highflow ventilator or any other ventilator.

The system may alternatively or additionally be characterized in that it comprises a sensor for detection of activity. Such a sensor can, for example, be an acceleration sensor or else a motion sensor. Said sensor is attached to the body (e.g., wrist and/or chest) and/or is a sensor in a mobile device comprising a data interface (e.g., smartphone) and/or even a sensor in a bed, via which the daily duration of rest can be captured. Such a sensor for detection of activity can also be combined with further sensors, for example for monitoring of temperature. In some embodiments, the activity sensor may also be permanently installed in the room in which, for example, the ventilator is used or the patient is treated.

The system may, for example, evaluate the activity of the patient on the basis of the signals of the activity sensor and, if necessary, trigger an alarm. For example, an alarm can be triggered when the activity of the patient is very low and/or non-existent over a defined period. For example, the activity sensor may be combined with a thermal imaging camera. Also, the system may, in some embodiments, be capable of comparing the activity registered by the activity sensor with activity recordings of the patient in order to establish an increased or decreased activity. Moreover, the system may, in some embodiments, be configured to assess activity on the basis of other sensor signals, for example temperature and/or respiratory gas sensors, and also possible analyses of said sensor signals.

Alternatively or additionally, the system may also be characterized in that the system comprises a pulse oximetry sensor which can, inter alia, determine the oxygen saturation of the blood, for example via light absorption and/or light remission/reflection. From the measurement values of the pulse oximetry sensor, the processing unit of the system can, for example, infer the health status of the patient. Generally, an oxygen saturation between 95% and 100% is considered adequate, whereas, for example, values of below 85% oxygen saturation may indicate a health problem. Such a sensor may be capable of measuring oxygen saturation and also pulse rate. A temperature sensor can also be combined with a pulse oximetry sensor. In addition to indications regarding possible diseases or regarding the state of the user or the patient, it is also possible in some embodiments to infer the quality of ventilation from the signals of the pulse oximetry. In some embodiments, the ventilation settings or parameters are adjusted on the basis of oxygen saturation and, optionally, temperature or other sensor signals. Said adjustment can, inter alia, even be done automatically.

Additionally or alternatively, the system may comprise at least one sensor for analysis of exhaled air with regard to general biomarkers, for example for inflammatory reactions or else for specific pathogens. Optionally, an analysis of temperature via the exhaled air may also possible. To this end, in some embodiments of the system, the sensor or the sensors are installed in the region of the patient interface. The sensor for analysis of exhaled air can, for example, also be arranged in the ventilator or between the ventilator and the patient interface if the exhaled air is being conducted in the direction of the ventilator. Also, the sensor for analysis of exhaled air can be arranged away from the ventilator and, advantageously, in connection with the patient interface.

The system may alternatively or additionally be characterized in that the system comprises at least one unit or at least one sensor for carrying out a “point-of-care” analysis of particular body extracts, excretions and/or fluids. In this connection, said extracts, fluids and/or excretions can, for example, be urine, blood, saliva, sputum, hair, nails and others. What can be examined is especially an analysis with respect to temperature, color spectrum, liquid content or particular ingredients.

In some embodiments, the ventilator or the ventilation settings can be automatically adjusted to the results of the analysis of the extracts/fluids/excretions. An analysis of the body extracts, excretions and/or fluids may, for example, be carried out by the analysis of test strips by an appropriate sensor. Such a sensor may, for example, be designed to detect the color change in individual test fields of a test strip which was previously contacted with the body extracts, excretions and/or fluids. The processing unit of the system may, for example, detect infections or indications of infections on the basis of the detection of the color changes or check whether the color change in the test strip is representative of an infection.

In some embodiments, the at least one sensor may be a biochemical sensor which is designed and configured to directly analyze or measure body extracts, excretions and/or fluids for particular ingredients. In some embodiments, the system may, for example, also be configured such that body extracts, excretions and/or fluids can be directly removed and/or collected by the system. For example, what can be mounted in the region of the patient interface or the ventilation tube to collect sputum from the patient—for example by coughing—is an appropriate collection unit which moreover has an appropriate sensor or multiple sensors configured for detection of infection. As already previously described in connection with a temperature sensor inter alia, it may be possible to initiate particular actions on the basis of the sensor signals, such as, for example, notices to the patient, generation of an alarm signal or triggering of a signal, saving/transmission of the data by telemonitoring, etc.

The signals of the above-described sensors can be evaluated in various alternative or additional embodiments of the system and detect an infection from defined threshold values being exceeded or fallen short of. In this connection, the processing unit of the ventilator may be configured and designed such that what can be detected is whether a sensor has been connected and what kind of sensor the sensor is or what kind of sensor data are being transmitted. In some embodiments, the processing unit may moreover be configured and designed to compare the sensor signals with alarm thresholds and, on the basis thereof, to generate a query of the state of the patient and/or to output appropriate recommended actions to the patient via an end-user interface. Alternatively or additionally, appropriate data may also be sent to a remote alarm as a result of the alarm threshold being exceeded or fallen short of.

Irrespective of the kind of sensor or sensors, what is calculated in some embodiments for each sensor signal is an index value which at least contains whether the sensor signal is representative of an infection. The respective index values of the individual sensor signals can, for example, be added up or totalized via another algorithm in order to calculate a total index. For said total index, it may be possible to store further threshold values which trigger various actions when they are exceeded, such as, for example, initially a simple notice to the patient and various alarm levels. In some embodiments, data transmission, for example by telemonitoring, may also be configured.

In some exemplary embodiments, various rules, for example so-called “fuzzy rules”, may be defined, whereby the sensor signals are linked to one another and a possible infection or the probability of an infection is ascertained on the basis of said linked sensor signals. In particular, it may be possible to this end to link the sensor signals to the respiratory rate determined via the ventilator. For example, a greatly increased respiratory rate in combination with at least one sensor signal deviating from a value defined as normal range may clearly point to an infection.

Furthermore, in some embodiments, sensor signals may also be linked to one another via a decision tree in order to determine an infection or the probability of an infection. Various sensor signals may thus, for example, be weighted differently. Also, branching of the individual sensor signals in a decision tree can, for example, be created by a machine-learning method.

In some embodiments, switch-on of further sensors may be triggered in accordance with a sensitive preliminary suspicion of an infection or the patient/user is prompted to switch on further sensors because of said preliminary suspicion. For example, an initial suspicion of an infection may be triggered by a slightly increased respiratory rate, for example at least 2 breaths per minute on average. Said initial suspicion may, for example, then bring about a notice to the patient, that further sensors, for example pulse oximeter or thermometer, are to be used. Further indicators in relation to the presence of an infection may, for example, also be determined from the service life of the ventilator or generally unusual fluctuations in respiratory rate and/or volume.

The dependent claims and also the description relate to various mutually independent, advantageous developments of the present invention, the features of which can, within the scope of what is technically meaningful, be combined freely with one another by a person skilled in the art. In particular, this also applies beyond the boundaries of the various claim categories.

### BRIEF DESCRIPTION OF THE DRAWING

Further features and advantages of the invention will become clear from the following description of non-limiting illustrative embodiments of the invention, which are explained in more detail below with reference to the only drawing in which the FIGURE shows a system for the noninvasive measurement of the body temperature during ventilation.

### DETAILED DESCRIPTION OF EXEMPLARY EMBODIMENT

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawing making apparent to those of skill in the art how the several forms of the present invention may be embodied in practice.

The only FIGURE shows a system 10 for the noninvasive measurement of the body temperature during ventilation, comprising at least one sensor 2, a ventilator 1, an interface 3 between ventilator and temperature sensor, a processing unit 4, a data storage unit 5, a user interface 6, an interface 7 to a remote alarm, a remote alarm 8, a ventilation tube 9 having a patient interface 19, and at least one respiratory gas sensor 11. The ventilator is a clinical ventilator or a ventilator for domestic ventilation or CPAP or bilevel therapy or a highflow ventilator or any other ventilator.

The ventilator is configured and designed to identify phases of inspiration and expiration by means of the respiratory gas sensor 11 and to convey respiratory gas during inspiration and expiration, and the system is configured to ascertain a sensor signal by means of the sensor 2 and the processing unit 4, at least in phases during inspiration and expiration, and to check whether the sensor signal is representative of body temperature and to process and/or save such body temperature signals and to compare them with saved rules or threshold values.

To sum up, the present invention provides:
1. A system 10 for the noninvasive detection of an infection during ventilation, which system comprises at least one sensor 2 configured for the detection of an infection, a ventilator 1, an interface 3 between ventilator and sensor 2, a processing unit 4, a data storage unit 5, a user interface 6, an interface 7 to a remote alarm, a remote alarm 8, a ventilation tube 9 having a patient interface 19, and at least one respiratory gas sensor 11, the ventilator being configured to identify the pressure and/or flow of respiratory gas by means of the respiratory gas sensor 11 and to convey pressurized respiratory gas and the system being configured to ascertain a sensor signal by means of the sensor 2 and the processing unit 4, at least in phases during ventilation, and to check whether the sensor signal is representative of an infection.
2. The system according to item 1, wherein the sensor 2 is a temperature sensor which detects a rise in body temperature as an infection when defined threshold values are reached or exceeded.
3. The system according to item 1 or item 2, wherein the at least one temperature sensor 2 has connected thereto a transmitter unit for transmitting measured temperature values.
4. The system according to at least one of the preceding items, wherein at least one temperature sensor 2 or sensor 2 is formed by an optical sensor.
5. The system according to at least one of the preceding items, wherein at least one temperature sensor 2 or sensor 2 is provided in the tube or the patient interface for measurement of the temperature of inspiration flow and/or expiration flow.
6. The system according to at least one of the preceding items, wherein at least one temperature sensor 2 or sensor 2 is arranged adjacent to the body or skin of the patient.
7. The system according to at least one of the preceding items, wherein an activity sensor 14 is provided in addition to the temperature sensor 2 or sensor 2.
8. The system according to at least one of the preceding items, wherein the transmission of sensor data via the interface 3 takes place at least daily, preferably at least hourly, particularly preferably at least 1× per minute.
9. The system according to at least one of the preceding items, wherein the processing unit 4 is designed and configured to detect whether a sensor signal is present and whether a sensor signal represents a temperature signal.
10. The system according to at least one of the preceding items, wherein the processing unit 4 is designed and configured to process the temperature signal, for example by conversion of the units, smoothing, . . . , and to save it in the data storage unit 5.
11. The system according to at least one of the preceding items, wherein the processing unit 4 is designed and configured to compare the temperature signal with stored alarm thresholds, said alarm thresholds representing an excessively high temperature or low temperature or the loss of the data connection or a deterioration of the sensor or an expiration of the service life of the sensor or excessively low battery level of the sensor.
12. The system according to at least one of the preceding items, wherein the processing unit 4 is designed and configured to compare the temperature signal with stored alarm thresholds and, in the event of an alarm threshold for increased body temperature being exceeded, the processing unit 4 is designed and configured to control at least one ventilation parameter 17 of the ventilator such that the result is a ventilation matched to the increased body temperature, thus ensuring an optimized relief or support for the patient.
13. The system according to at least one of the preceding items, wherein the processing unit 4 is designed and configured to compare the temperature signal with stored alarm thresholds and, in the event of an alarm threshold for an increased or decreased body temperature being exceeded (temperature alarm), the processing unit 4 is designed and configured to request, in an automated manner, feedback from the patient, said feedback being a digital response from the patient that represents a current state.
14. The system according to at least one of the preceding items, wherein the processing unit 4 is designed and configured to compare the temperature signal with stored alarm thresholds and, in the event of an alarm threshold for an increased or decreased body temperature being exceeded (temperature alarm), the processing unit 4 is designed and configured to output recommended actions to the patient via the end-user interface or a loudspeaker.
15. The system according to at least one of the preceding items, wherein the processing unit 4 is designed and configured to form statistics, for example containing max/min/mean values/proportion with value >x for defined time intervals, for example per hour or per day, and to save them in the data storage unit 5.
16. The system according to at least one of the preceding items, wherein the processing unit 4 is designed and configured to process signals of the sensor 2 and signals of the respiratory gas sensor 11 and signals of the activity sensor 14 and to plausibilize signals of the sensor 2 by a comparison with signals of the respiratory gas sensor 11 and/or signals of the activity sensor 14.
17. The system according to at least one of the preceding items, wherein the data storage unit 5 is designed and configured to record, in a retrievable manner, the course of the sensor signal and preferably also the statistics which were calculated and alarms which occurred.
18. The system according to at least one of the preceding items, wherein the user interface 6 is designed and configured to generate feedback as to whether a sensor has been connected correctly and is providing values or not, preferably in the form of a graphical and acoustic output, for example warning tone in the event of an alarm.
19. The system according to at least one of the preceding items, wherein the interface to the remote alarm 7 is designed and configured to conduct either data only (remote alarm has its own power source) or data and power in relation to the remote alarm 8.
20. The system according to at least one of the preceding items, wherein the interface to the remote alarm 7 is configured and designed for at least daily, preferably at least hourly, preferably at least 1× per minute, transmission; preferably also triggered by the occurrence of an alarm.
21. The system according to at least one of the preceding items, wherein the system further comprises a humidifier 15 which is configured and designed to warm and humidify respiratory gas, account being taken of body temperature signals of the temperature sensor 2 at least from time to time or in part for the control of the humidifier in order to warm and humidify the respiratory gas according to the body temperature signal.
22. A ventilator configured and designed for use in a system or with a system according to at least one of the preceding items.

### LIST OF REFERENCE NUMERALS

Ventilator 1
Sensor or temperature sensor 2, body
Interface 3 between ventilator and sensor
Processing unit 4
Data storage unit 5
User interface 6
Interface 7 to the remote alarm
Remote alarm 8
Ventilation tube 9 having a patient interface 19
Respiratory gas sensor 11
Inspiration 12
Expiration 13
Activity sensor 14
Humidifier 15
End-user interface 16
Ventilation parameter 17
Alarm thresholds 18
Patient interface 19
Temperature sensor 20, surroundings

What is claimed is:

1. A system for noninvasive detection of an infection during ventilation, wherein the system comprises at least one sensor configured for detection of an infection, a ventilator, an interface between the ventilator and the sensor, a processing unit, a data storage unit, a user interface, an interface to a remote alarm, a remote alarm, a ventilation tube having a patient interface, and at least one respiratory gas sensor, the ventilator being configured to identify pressure and/or flow of respiratory gas by means of the the at least one respiratory gas sensor and to convey pressurized respiratory gas and the system being configured to ascertain a sensor signal by means of the sensor and the processing unit, at least in phases during ventilation, and to check whether the sensor signal is representative of an infection.

2. The system of claim 1, wherein the sensor is a temperature sensor which detects a rise in body temperature as an infection when defined threshold values are reached or exceeded.

3. The system of claim 2, wherein the at least one temperature sensor has connected thereto a transmitter unit for transmitting measured temperature values.

4. The system of claim 1, wherein at least one temperature sensor or sensor is an optical sensor.

5. The system of claim 1, wherein at least one temperature sensor or sensor is provided in the tube or the patient interface for measurement of the temperature of inspiration flow and/or expiration flow.

6. The system of claim 1, wherein at least one temperature sensor 2 or sensor 2 is arranged adjacent to a body or skin of a patient.

7. The system of claim 1, wherein an activity sensor is provided in addition to the temperature sensor or sensor.

8. The system of claim 1, wherein the transmission of sensor data via the interface takes place at least daily.

9. The system of claim 1, wherein the processing unit is designed and configured to detect whether a sensor signal is present and whether a sensor signal represents a temperature signal.

10. The system of claim 1, wherein the processing unit is designed and configured to process the temperature signal, and to save it in the data storage unit.

11. The system of claim 1, wherein the processing unit is designed and configured to compare a temperature signal with stored alarm thresholds, the alarm thresholds representing an excessively high temperature or low temperature or a loss of the data connection or a deterioration of the sensor or an expiration of a service life of the sensor or an excessively low battery level of the sensor.

12. The system of claim 1, wherein the processing unit is designed and configured to compare a temperature signal with stored alarm thresholds and, in the event of an alarm threshold for increased body temperature being exceeded, is designed and configured to control at least one ventilation parameter of the ventilator such that the result is a ventilation matched to the increased body temperature.

13. The system of claim 1, wherein the processing unit is designed and configured to compare a temperature signal with stored alarm thresholds and, in the event of an alarm threshold for an increased or decreased body temperature being exceeded (temperature alarm), is designed and configured to request, in an automated manner, feedback from a patient, said feedback being a digital response from the patient that represents a current state and/or is designed and configured to output recommended actions to the patient via an end-user interface or a loudspeaker.

14. The system of claim 1, wherein the processing unit is designed and configured to form statistics and to save them in the data storage unit.

15. The system of claim 7, wherein the processing unit is designed and configured to process signals of the sensor and signals of the at least one respiratory gas sensor and signals of the activity sensor and to plausibilize signals of the sensor by a comparison with signals of the respiratory gas sensor and/or signals of the activity sensor.

16. The system of claim 1, wherein the data storage unit is designed and configured to record, in a retrievable manner, a course of the sensor signal.

17. The system of claim 1, wherein the user interface is designed and configured to generate feedback as to whether a sensor has been connected correctly and is providing values or not.

18. The system of claim 1, wherein the interface to the remote alarm is designed and configured to conduct either data only or data and power in relation to the remote alarm.

19. The system of claim 1, wherein the interface to the remote alarm is configured and designed for at least daily transmission.

20. A ventilator, wherein the ventilator is configured and designed for use in a system or with a system of claim 1.
